(19) United States
(12) Patent Application Publication
(10) Pub. No.: US 2014/0276696 A1
(43) Pub. Date: Sep. 18, 2014

(54) THREADED LEAD EXTRACTION DEVICE

(71) Applicant: THE SPECTRANETICS CORPORATION, Colorado Springs, CO (US)

(72) Inventor: Blaine Andrew Schneider, Colorado Springs, CO (US)

(73) Assignee: The Spectranetics Corporation, Colorado Springs, CO (US)

(21) Appl. No.: 13/828,491

(22) Filed: Mar. 14, 2013

(51) Int. Cl. A61B 17/3207; A61B 18/24

(52) U.S. Cl. CPC A61B 17/320758; A61B 18/24
USPC 606/15; 606/129

(57) ABSTRACT

Methods and devices for separating an implanted object, such as a lead for a mechanical pacemaker or a cardioverter defibrillator, from tissue surrounding such object in a patient's vasculature system. The methods and devices comprise a means for applying tension to the object at the working end of a catheter to reduce the catheter's likelihood of bending or kinking the lead during extraction.

### FIELD OF THE DISCLOSURE

The present disclosure relates generally to devices, methods and systems for separating tissue in a patient, and more specifically, to devices for separating tissue attached to implanted objects, such as leads, in a patient and removing such objects.

### BACKGROUND

Although there may be more, there are generally at least two primary types of cardiac conduction devices (CCDs). Those two primary types of CCDs are mechanical pacemakers and implantable cardioverter defibrillators. A mechanical pacemaker is an electronic device that produces small bursts of electrical energy to the heart, when needed, to increase the heart rate during period(s) when the heart's natural electrical activity is slower than desirable. Alternatively, implantable cardioverter-defibrillators stop dangerously rapid heart rhythms by delivering a large electric shock to the heart to prevent cardiac arrest.

Implantable cardioverter-defibrillators typically include particular types of coils that provide the electric shock. The leads are generally placed within the region of the brachiocephalic vein-superior vena cava junction and in the right ventricle positioned so that the shock coils are located in the region of the brachiocephalic vein-superior vena cava junction and in the right ventricle. An implantable cardioverter-defibrillator is capable of sensing the heart's rhythm, and in the event it senses a particular type of rhythm, such as tachyarrhythmia, the implantable cardioverter-defibrillators sends a relatively large shock to the heart.

For the CCDs to work effectively, the leads are preferably in contact with heart tissue. As illustrated in FIG. 1, aloud 104 typically passes through a vein under the collarbone to the innominate vein, past the superior vena cava (“SVC”) 116, and into the right atrium 120 of the heart 100. The distal portion 108 of the lead 104 then enters the right ventricle 124 and attaches to the heart via a fixation mechanism 112, such as a small screw and/or hooks at the end. In certain instances, a lead may be attached to the outside of the heart.

Within a relatively short time after a lead is implanted into the body, the body's natural healing process forms scar tissue along the lead and possibly at its tip, thereby fastening it even more securely in the patient's body. Although leads are designed to be implanted permanently in the body, occasionally these leads must be removed, or extracted. Leads may be removed from patients for numerous reasons, including but not limited to, infections, lead age, and lead malfunction.

Removal or extraction of the lead may be difficult. As mentioned above, the body's natural healing process forms scar tissue over and along the lead, and possibly at its tip, thereby encasing at least a portion of the lead and fastening it even more securely within the subject's body. In addition, the lead and/or tissue may become attached to the vasculature wall. Both results may, therefore, increase the difficulty of removing the leads from the subject's vasculature. This potential difficulty may also be exacerbated when coiled leads are embedded in and/or attached to the subject's vasculature.

A variety of tools have been developed to make lead extraction safer and more successful. Current lead extraction techniques include mechanical traction, mechanical devices, and laser devices. Mechanical traction may be accomplished by inserting a locking stylet into the hollow portion of the lead and then pulling the lead to remove it. An example of such a lead locking device is described and illustrated in U.S. Pat. No. 6,167,315 to Coe et al., which is hereby incorporated herein by reference in its entirety for all that it teaches and for all purposes.

A device used to extract leads may include a flexible tube or sheath or catheter that passes over the lead and/or the surrounding tissue. The catheter generally has a working tip, such as a sharp blade to cut the tissue or a plurality of laser emitters, which are often the distal ends of optical fibers within a laser catheter, to ablate the tissue. If a cutting blade is used, it generally cooperates with a surrounding sheath to separate the scar tissue from the lead and/or other scar tissue. In some cases, the cutting blade and sheath may also separate the tissue itself from the lead. Once the lead is separated from the surrounding tissue and/or the surrounding tissue is separated from the remaining scar tissue, a sheath (or other mechanical device) may be used to remove the lead from the patient's vasculature. Examples of such mechanical cutting catheters or lead removal devices may include those illustrated and discussed in U.S. Publication Nos. 2008/0154293 and 2008/0154296 both of which are incorporated herein by this reference in their entirety. Another examples of such a devices and methods used to extract leads is described and illustrated in U.S. Pat. No. 5,651,781 to Grace, which is hereby incorporated herein by reference in its entirety for all that it teaches and for all purposes.

Additionally, examples of laser ablation catheters or sheaths may include in U.S. Pat. Nos. 5,456,680 and 5,643,251 both of which are incorporated herein by this reference in their entirety. Furthermore, Spectranetics Corporation, the assignee of this disclosure, sells a laser sheaths under the trade names GlideLight™ and SLS II. Laser sheaths, such as these, have a plurality of laser emitters at its distal tip.

Regardless of whether the surgeon or clinician is using a sharp blade or laser ablation sheath to separate the lead from the scar tissue, it is helpful to increase co-axial alignment between the lead and the working tip, particularly when the leads are located in, and/or attached to a structurally-weak portion of the vasculature. For instance, typical leads in a human may pass through the innominate vein, past the superior vena cava (“SVC”), and into the right atrium of the heart. Tissue growth occurring along the SVC and other locations along the innominate vein may increase the risk and difficulty in extracting the leads from such locations, particularly when the vein(s)′ walls are thin. These risks are potentially magnified if co-axial alignment is not maintained between the lead and the working tip. Proper alignment reduces the likelihood that the working tip will cut into the lead itself, which is often referred to as a snowplow. Proper alignment also reduced the likelihood that the working tip will take a large so-called “bite” out of the surrounding tissue. Tissue growth may also occur at other challenging locations within a patient's vasculature. Co-axial alignment between the lead the working tip of the catheter or sheath may also be beneficial in these instances, particularly when extracting coiled leads.

### SUMMARY

Accordingly, there is a need for a device, method and/or system such as a surgical device that has the capability to facilitate and maintain improved co-axial alignment between the lead and the working tip of the catheter (or sheath) when extracting leads, particularly curved shaped leads including coiled portions theof. For example, it may be desirable to create traction between the lead and the catheter (or sleeve) at its distal and working end. Providing such traction, force, tension, etc. at the working end of the catheter (or sleeve) increases the likelihood of maintaining co-axial alignment between the lead and the working tip of the catheter (or sheath), which reduces the potential for bending or kinking of the lead, snowplowing, and ablating excessive amounts of surrounding tissue.

A method of removing an implanted object, such as a lead, from a subject's vasculature, may comprise the steps of (i) advancing a catheter over a lead having a coil, the catheter comprising (a) an outer jacket, (b) an inner band located radially inwardly of the outer jacket, the inner band having a distal end creating a lumen extending proximally from its distal end, and at least a portion of the inner band comprising a threaded portion configured to matingly engage the coil, and (c) a plurality of optical emitters located at the distal end of the inner band, and (ii) rotating the inner band in a clockwise and/or counter-clockwise direction to engage the coil, thereby substantially aligning a longitudinal axis of the coil with a longitudinal axis of the catheter.

A device for removing an implanted object may include a catheter having a distal end, a lumen extending proximally from the distal end, means for separating tissue adjacent to a lead having a coil, the means for separating the tissue being located at the distal end of the catheter and creating an orifice for the lumen, and an inner band located within the distal portion of the lumen, the inner band comprising a threaded portion configured to matingly engage the coil as the inner band rotates. The means for separating the tissue may include a plurality of optical fibers located within the catheter and terminating around at least a portion of the orifice and/or a cutting blade attached thereto and extending from the distal end of the catheter.

The present disclosure incorporates a threaded portion within the interior of the catheter, potentially at the distal tip of the catheter, to mate with the thread-like configuration of the coiled lead and apply a tensile force to the lead upon rotation of the catheter. The tensile force maintains tension between the lead and the catheter as the means for separating the tissue from the lead and potentially reduces the likelihood of the lead bending or kinking as the means for separating tissue and/or catheter advances over the lead.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “computer-readable medium” as used herein refers to any storage and/or transmission medium that participate in providing instructions to a processor for execution. Such a medium is commonly tangible and non-transient and can take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

The terms “determine”, “calculate” and “compute,” and variations thereof, as used herein, are used interchangeably and include any type of methodology, process, mathematical operation or technique.

A “lead” is a conductive structure, typically an electrically insulated coiled wire. The electrically conductive material may be any conductive material, with metals and intermetallic alloys common. The outer sheath of insulated material is biocompatible and bio stable (e.g., non-dissolving in the body) and generally includes organic materials such as polyurethane and polyimide. Lead types include, by way of non-limiting example, epicardial and endocardial leads. Leads are commonly implanted into a body percutaneously or surgically.

The term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

The term “module” as used herein refers to any known or later developed hardware, software, firmware, artificial intelligence, fuzzy logic, or combination of hardware and software that is capable of performing the functionality associated with that element. Also, while the disclosure is presented in terms of exemplary embodiments, it should be appreciated that the individual aspects of the disclosure can be separately claimed.

A “surgical implant” is a medical device manufactured to replace a missing biological structure, support, stimulate, or treat a damaged biological structure, or enhance, stimulate, or treat an existing biological structure. Medical implants are man-made devices, in contrast to a transplant, which is a transplanted biomedical tissue. In some cases implants contain electronics, including, without limitation, artificial pacemaker, defibrillator, electrodes, and cochlear implants. Some implants are bioactive, including, without limitation, subcutaneous drug delivery devices in the form of implantable pills or drug-eluting stents.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic view of a lead deployed in a patient's heart;

FIG. 2 is an elevation view of a coiled lead according to an embodiment of the present disclosure;

FIG. 3 is an elevation view of a coiled lead according to an alternate embodiment of the present disclosure;

FIG. 4A is an elevation view of a coiled lead according to an embodiment of the present disclosure embedded within scar tissue in a blood vessel;

FIG. 4B is an elevation view of an embodiment of a surgical device, such as a catheter, according to an embodiment of the present disclosure.

FIG. 5 is a perspective view of a distal end portion of a laser catheter according to one embodiment of the present disclosure;

FIG. 6 is an end view of a distal end portion of a laser catheter according to one embodiment of the present disclosure;

FIG. 7 is an elevation view of a distal end portion of a laser catheter according to one embodiment of the present disclosure;

FIG. 8 is a schematic of a control system according to one embodiment of the present disclosures;

FIG. 9 is a schematic of a control algorithm according to one embodiment of the present disclosures; and

FIG. 10 is surgical device including a pressure indicator according to one embodiment of the present disclosures.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Embodiments according to this disclosure provide a surgical device that can be deployed safely within a vascular system of a patient and separate implanted objects, such as leads and particularly coiled leads from a subject's vasculature system. With reference to FIG. 2, an exemplary lead 200 having a coil or coiled portion 204 and an optional a layer of electrical insulation 208 is depicted. The coil 204 includes multiple strands 212 placed closely adjacent one another and wound in a spiral or helical fashion. The coil 204 is wound in a configuration such that upon each revolution of a set 216 of strands 212, a space 218 is created therebetween, thereby creating a thread-like pattern. The lead depicted in FIG. 2 is typically referred to as a unipolar configuration because the coil 204 is the cathode that works in cooperation with a separately located anode (not shown).

Other configurations of coiled leads may include a bipolar configuration, such as that depicted in FIG. 3. The lead 300 illustrated in FIG. 3 includes two coils 304, 312, wherein the inner coil 304 acts as the cathode, and the outer coil 312 acts as the anode. The lead 300 may preferably include an inner insulation layer 308 between the inner coil 304 and outer coil 312. Additionally, the outer coil 312 may be surrounded by an outer layer 316 of electrical insulation. Similar to FIG. 2, the inner coil 316 includes multiple strands placed closely adjacent one another and wound in a spiral or helical fashion such that a thread-like pattern is created. The outer coil 312 also has a similar construction. The outer coil 312 includes multiple strands 320 placed closely adjacent one another and wound in a spiral or helical fashion such that upon each revolution of a set 324 of strands 320, a space 326 is created therebetween, thereby creating a thread-like pattern. Depending upon the desired performance of the coils, the number of strands per set, the size and configuration of the strands, the space between the sets of strands and other characteristics of the coils may change. Those of skill in the art will understand how to make and use the disclosed aspects, embodiments, and/or configurations after understanding the present disclosure to adjust the configurations of the coils. All such configurations within the knowledge of one skilled in the art are considered within the scope of this disclosure.

The present disclosure takes advantage of the thread-like configuration of coil(s) by incorporating a mating threaded section within the distal end of the catheter used to separate the tissue from the lead. Upon the coil mating with the distal end of the catheter, a tensile force is applied to the lead, thereby maintaining tension on the lead as a means for separating tissue located at the distal end of the catheter from the lead. Applying tension to the lead at or near the point of tissue separation reduces the likelihood of the lead bending or kinking as the means for separating tissue from the lead as the catheter advances.

With reference to FIG. 4A, there is shown a coil 204 of FIG. 2 embedded within scar tissue 408 of a blood vessel in a subject's vascular system. With reference to FIG. 4B, there is shown a catheter 800 having a threaded portion within the distal portion of the lumen that matingly engages the thread-like (or grooved) pattern of the coil such that as the catheter, particularly the tissue separating means (not shown), rotates and advances over the coil 204, the coils 204 (and lead) remain taught.

With reference to FIGS. 5, 6 and 7, an embodiment of the present disclosure of a laser catheter used to separate tissue from a coiled lead may include a plurality of optical fibers 504 located between an outer jacket (or sleeve) 508 and an inner band 512. The outer jacket 508 comprises a flexible assembly with the ability to resist user-applied forces such as torque, tension, and compression. The proximal end (not shown) of the catheter 500 is attached to a fiber optic coupler (not shown) and includes an outer jacket, inner band and a plurality of optical fibers similar to the configuration and orientation of such components depicted in FIGS. 5 and 6. The distal end 516 of the catheter 500 may comprise a tapered outer band 520, which is attached to the distal end of the outer jacket 508. The plurality of optical fibers 504 act as laser emitters, and the inner band 512 creates an orifice that provides an entrance to an inner lumen 14 that may be connected to an optional aspiration system (not shown). The energy emitted by the laser emitters 504 cuts, separates, and/or ablates the scar tissue, plaque build-up, calcium deposits and other types of undesirable lesion or bodily material within the subject's vascular system in a pattern substantially similar to that of the cross sectional configuration of the laser emitters 10. In this particular embodiment of the present disclosure, the optical fibers/laser emitters 504 are arranged generally concentrically about the orifice, which are aligned with the longitudinal axis of the laser catheter 500. Also provided substantially concentric with and interior to the laser emitters 504 (and optical fibers) is an inner lumen 524, which provides a potential conduit or passageway for translocation of materials cut or ablated by the laser emitters 504.

As the energy emitted by the laser emitters 504 contacts the undesirable bodily material within the subject's vascular system, it separates and cuts such material in a generally concentric configuration. In other words, one of ordinary skill in the art may refer to this technique as coring. And if the bodily material that is cut is substantially solid, it will appear as generally cylindrically looking core or plug. Although FIGS. 5 and 6 illustrate the laser emitters 504 in a generally concentric configuration, those skilled in the art will appreciate that there are numerous other ways and configurations in which to arrange a plurality of laser emitters. Additionally, although these three figures illustrate an outer jacket 508 and an inner band 512, those of skill in the art will appreciate that distinct components need not be used, and the optical fibers may be encapsulated within a single sleeve having a lumen. Accordingly, FIGS. 5 and 6, as well as FIG. 7 discussed below, are not intended to represent the only way that a laser catheter may be configured and constructed, and all such configurations and constructions are within the knowledge of one skilled in the art are considered within the scope of this disclosure.

FIG. 7 is a cross-sectional perspective view of a laser catheter according to one embodiment of the present disclosure. A flexible distal tip 700 is provided, the distal tip 700 comprising a central or inner lumen 524 provided substantially concentric with one or more annular arrays of optical fibers 504 and an outer jacket 508. An inner band 512 is provided at a far distal end of the tip 700. The inner band 512 comprises a proximal end, a distal end, an interior surface, and an exterior surface. When placed within the distal tip 700 of the laser catheter 500, the distal end of the inner band 512 is substantially aligned (or flush) with the far distal end of the tip 700. The inner band 512 may be attached to the catheter via numerous means known to one of ordinary skill in the art. For example, the dimension of the exterior diameter (or circumference) of the inner band 12 may be slightly greater than the diameter (or circumference) of the lumen at the distal tip of the catheter such that the inner band is press fit into the distal tip 700 of the catheter 500. Additionally, the inner band 512 may be attached to the lumen by various known adhesives.

The inner band 512 comprises a spiral or helically shaped structure 532 extending from its distal end to its proximal end continuously or for portions thereof. The helical structure 532 may comprise a polymer extrusion or metal insert extending radially inwardly from an inner diameter of the inner band 512 and/or along a length of the lumen 524 in a helical or spiral manner. Alternatively, the helical structure 532 may be provided as a recessed grooved feature along the inner band 512 or lumen 524 of the catheter 500. The helical structure 532 or recessed feature within its interior surface, is configured to create a thread-like pattern than can mate with the thread-like pattern of the coil in the lead. As the catheter 500 rotates, the thread(s) 532 of the inner band 512 mate with the thread(s) or thread-like pattern of the coil.

Although FIG. 7 illustrates the threads 532 only in the inner band 512, which is at the distal tip 700 of the catheter, those of skill in the art will appreciate that the threads 532 may extend into the lumen or merely be included in the lumen, as well be placed in various locations within the catheter. Accordingly, FIG. 7 is not intended to represent the only thread-like configuration of the inner band 512 to engage the tread-like pattern of a coiled lead, and all such configurations and constructions are within the knowledge of one skilled in the art are considered within the scope of this disclosure.

The present disclosure is not limited to a thread-like configuration of the inner band and/or lumen being used to only engage the tread-like pattern of a coiled lead. The present disclosure also envisions a thread-like configuration of the inner band and/or lumen being used to engage the outer jacket and/or insulation of the lead. Stated differently, the thread-like configuration of the inner band and/or lumen can also engage and/or cut through the outer jacket of the lead to engage the inner coils along part or all of the lead. In such embodiments, the helical structure may have hardness to engage and/or cut through the outer jacket, such as a hardness greater than that of the outer jacket. For example, the helical structure may be constructed of a biocompatible metal with an abrasive coating, such as a diamond coated metal. Similarly, the helical structure may also be constructed of a biocompatible polymer that has sufficient rigidity and hardness to engage and/or cut through the outer jacket.

The catheter 500 comprises a flexible distal end 700, the flexible distal end 700 being operable by a user. Although the clinician typically slides the catheter 500 over the lead (via the lumen) to perform a lead extraction procedure, the position of the distal end may optionally be controlled by one or more deflection means 528 which may include, but are not limited to, pullwires, shaping wires, and similar force-transmitting features controlled by a user at a user-proximal location of the device. Actuation of at least one deflection means 528 applies force to the distal tip 700, thus deflecting the distal tip 700 from a longitudinal axis of the remainder of the catheter device.

The inner band 512 or other threaded portions of the lumen in the catheter may be coupled to a pressure sensor (not shown) that is capable of determining the pressure of the inner band 512 (or other threaded catheter portions) created by rotating the catheter. The pressure created by the inner band 512 is transferred to the lead, particularly the coil, when mated with the threads 532 of the inner band 512. Accordingly, the pressure sensor is indicative of the pressure (and/or force) applied to the coil of the lead. The pressure sensor of this disclosure is not limited to a particular type and may include any type, such as a piezoresistive sensor, capacitive sensor, electromagnetic sensor, piezoelectric sensor, optical sensor, and a potentiometric sensor.

Referring to FIG. 8, there is depicted an embodiment of a control system 800 for monitoring the pressure sensor. The control system 800 includes the pressure sensor 804 in signal communication with a controller 808, which is, in turn, in signal communication with a user interface 812. The controller 808 executes one or more microprocessor executable control algorithms stored in a computer readable medium to receive catheter movement signals from the pressure sensor 804 and, based on the received pressure signals, provide alarm and other output to the user via user interface 812. The user interface can provide audible (e.g., sounds), tactile, and/or visual (e.g., lights) output to the user. Audible, tactile, and/or visual output relates to one or more of pressure and/or force applied to the catheter's distal tip and/or coiled lead within the body lumen.

An operation of the controller 808 will now be described with reference to FIG. 9. Operation commences when the controller 808 senses a stimulus in step 900. The stimulus, for example, can be the receipt of user input, receipt of a pressure signal from the pressure sensor 804, passage of time, and the like. In optional step 904, based upon the signal from the pressure sensor 804, the controller 808 determines a pressure and/or force applied at the catheter's distal tip and/or coiled lead. The controller 808 includes computer readable medium that may have predetermined thresholds corresponding low, medium and/or high pressures. In step 908, the sensed pressure is compared to the predetermined thresholds to determine whether the applied pressure is acceptable. The rule, for example, can be to provide audible, tactile, and/or visual output to the user, depending upon which threshold the pressure or force exceeds.

Accordingly, in step 908, the controller 908 determines, based on a comparison of a predetermined threshold to the pressure, whether applied pressure is acceptable. Regardless of whether the pressure is acceptable, the controller 808 proceeds to step 912 and optionally notifies the user accordingly. This process is continuously repeated, thereby continuously updating the user of whether additional pressure and/or rotation of the catheter is acceptable without breaking or harming the coil or lead.

Referring to FIG. 10, there is a depicted an embodiment of surgical device 1000 that includes a catheter 1004 and a handle 1008. The handle has an indicator 1012, which comprises a visual indicator 1012 user interface. The visual indicator 1012 includes a series of lights indicative the various pressure levels being applied to the lead. The embodiment depicted in FIG. 10, includes a green light 1016, a yellow light 1020, and a red light 1024 indicative of whether the applied pressure is acceptable, beginning to be unacceptable, and is unacceptable.

Although the indicator 1012 in FIG. 10 is located on the top of the proximal portion of the handle 1008, FIG. 10 is not intended to represent the only location and type of indicator that may be included in a serrated cutting surface. Depending upon the size and configuration of the surgical device, particularly its handle and actuating mechanism(s), those of skill in the art will understand how to make and use the disclosed aspects, embodiments, and/or configurations after understanding the present disclosure to adjust the location, size, configuration and/or type of indicator. All such configurations within the knowledge of one skilled in the art are considered within the scope of this disclosure. Additionally, visual indicators other than color (e.g., green, yellow, orange, red, etc.) of the light may be used, such as the brightness of the light and/or whether light remains constant or blinks (including the frequency of blinking) may be used to indicate the pressure, pressure conditions, pressure zones, etc.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

Presented herein are embodiments of a tissue separating device, system, and method. As described herein, the device(s) may be electrical, mechanical, electro-mechanical, and/or combinations thereof.

A number of variations and modifications of the disclosure may be used. It would be possible to provide for some features of the disclosure without providing others.

In some embodiments, the systems and methods of this disclosure may be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein may be used to implement the various aspects of this disclosure. Exemplary hardware that may be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing may also be constructed to implement the methods described herein.

The present disclosure, in various aspects, embodiments, and/or configurations, includes components, methods, processes, systems and/or apparatus substantially as depicted and described herein, including various aspects, embodiments, configurations embodiments, sub combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and/or reducing cost of implementation. All such configurations within the knowledge of one skilled in the art are considered within the scope of this disclosure.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Summary, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A catheter assembly comprising:
a catheter having a distal end and a lumen extending proximally from the distal end;
means for separating tissue adjacent to a lead having a coil, wherein the means for separating the tissue is located at the distal end of the catheter and creates an orifice for the lumen; and
an inner band located within the distal portion of the lumen, the inner band comprising a threaded portion configured to matingly engage the coil as the inner band rotates.

2. The catheter assembly of claim 1, wherein the means for separating tissue comprises a plurality of optical fibers located within the catheter and terminating around at least a portion of the orifice.

3. The catheter assembly of claim 1, wherein the plurality of optical fibers comprises at least one row of optical fibers located substantially concentric about the longitudinal axis of the catheter.

4. The catheter assembly of claim 1, wherein the plurality of optical fibers comprises two rows of optical fibers.

5. The catheter assembly of claim 1, further comprising an outer jacket, wherein the optical fibers are located between the outer jacket and the inner band.

6. The catheter assembly of claim 1, wherein the means for separating tissue comprises a cutting blade attached thereto and extending from the distal end of the catheter.

7. The catheter assembly of claim 6, wherein the means for separating tissue further comprises means for rotating the cutting blade.

8. The catheter assembly of claim 7, wherein the means for separating tissue further comprises means for extending the cutting blade as it is rotated.

9. The catheter assembly of claim 1, further comprising a pressure sensor coupled to the inner band.

10. The catheter assembly of claim 9, wherein the pressure sensor is at least one of a piezoresistive sensor, capacitive sensor, electromagnetic sensor, piezoelectric sensor, optical sensor, and a potentiometric sensor.

11. The catheter assembly of claim 9, further comprising a control system for monitoring the pressure sensor.

12. The catheter assembly of claim 11, further comprising an indicator that indicates a representation of pressure exerted by the inner band as it rotates and engages the coil.

13. The catheter assembly of claim 12, wherein the indicator is at least one of a visual indicator, tactile, indicator or an audible indicator.

14. A catheter configured to remove a lead from a subject's vasculature comprising:
an outer jacket;
an inner band located radially inwardly of the outer jacket, wherein the inner band has a distal end, wherein the inner band creates a lumen extending proximally from its distal end, wherein at least a portion of the inner band comprises a threaded portion configured to matingly engage a coiled portion of a lead; and
a plurality of optical fibers located between the outer jacket and the inner band, wherein the plurality of optical fibers terminate at about the distal end of the inner band.

15. The catheter assembly of claim 9, wherein the plurality of optical fibers comprises at least one row of optical fibers located substantially concentric about the longitudinal axis of the catheter.

16. The catheter assembly of claim 15, further comprising a pressure sensor coupled to the inner band.

17. The catheter assembly of claim 16, wherein the pressure sensor is at least one of a piezoresistive sensor, capacitive sensor, electromagnetic sensor, piezoelectric sensor, optical sensor, and a potentiometric sensor.

18. The catheter assembly of claim 16, further comprising a control system for monitoring the pressure sensor.

19. The catheter assembly of claim 18, further comprising an indicator that indicates a representation of pressure exerted by the inner band as it rotates and engages the coil.

20. A method of removing an embedded lead from a subject's vasculature, the method comprising the steps of:
advancing a catheter having over a lead having a coil, the catheter comprising:
an outer jacket;
an inner band located radially inwardly of the outer jacket, wherein the inner band has a distal end, wherein the inner band creates a lumen extending proximally from its distal end, wherein at least a portion of the inner comprises a threaded portion is configured to matingly engage the coil; and
a plurality of optical emitters located at the distal end of the inner band;
rotating the inner band in a clockwise and/or counter-clockwise direction to engage the coil, thereby substantially aligning a longitudinal axis of the coil with a longitudinal axis of the catheter.
